(19) United States
(12) Patent Application Publication
(10) Pub. No.: US 2024/0004540 A1
(43) Pub. Date: Jan. 4, 2024

(54) METHODS, APPARATUSES, AND COMPUTER-READABLE MEDIA FOR ENHANCING DIGITAL PATHOLOGY PLATFORM

(71) Applicant: Gestalt Diagnostics, LLC, Spokane, WA (US)

(72) Inventors: Roopam Kakoti, Spokane, WA (US); Dan Roark, Spokane, WA (US)

(21) Appl. No.: 18/469,278

(22) Filed: Sep. 18, 2023

Related U.S. Application Data
(63) Continuation-in-part of application No. 17/845,670, filed on Jun. 21, 2022.
(60) Provisional application No. 63/215,887, filed on Jun. 28, 2021.

(51) Int. Cl. G06F 3/04847; G06F 3/04817; G16H 30/40

(52) U.S. Cl. CPC G06F 3/04847; G06F 3/04817; G16H 30/40

(57) ABSTRACT

Techniques may include displaying a user interface for a digital pathology platform and displaying a slide panel on the user interface. A thumbnail of a digitized image of a specimen can be displayed. A settings button can be displayed. An operation can be received on the settings button. A settings window can be displayed in response to receiving the operation on the settings button. One or more setting operations can be received via the settings window. One or more interactive icons associated with the slide panel can be displayed based on the one or more setting operations via the settings window. The one or more interactive icons can indicate status associated with the slide panel.

### CROSS REFERENCE TO RELATED PATENT APPLICATIONS

This application is a continuation in part application of and claims priority to U.S. patent application Ser. No. 17/845,670, filed on Jun. 21, 2022, entitled “METHODS, APPARATUSES, AND COMPUTER-READABLE MEDIA FOR ENHANCING DIGITAL PATHOLOGY PLATFORM,” which claims the benefit of U.S. Provisional Patent Application No. 63/215,887, and filed Jun. 29, 2021, entitled “METHODS, APPARATUSES, AND COMPUTER-READABLE MEDIA FOR ENHANCING DIGITAL PATHOLOGY PLATFORM”, which is hereby incorporated by reference in its entirety.

### BACKGROUND

In pathology, the slide along with the microscope are important artifacts for disease diagnosis. The slide usually includes a label and a stained section of tissue from the original specimen of the patient. The label in turn may contain a barcode which assigns a unique identifier for the slide, patient identifying information (like name, Medical Record Number (MRN), etc., additional information such as stain, body part/specimen type, etc.).

Digital pathology is a sub-field of pathology that focuses on data management based on information generated from digitized specimen slides. Glass slides are converted into Whole Slide Images (WSI) that can be viewed by pathologists on a terminal device without being physically transported. Digital pathology platform is a dynamic, image-based environment that enables the pathologist to review, manage, and analyze pathology information generated from a digitized slide.

While rich data such as annotations may be added to the digitized slide displayed on the digital pathology platform, the information still does not enable the pathologist to know about the existence of such data without looking at the image. Since the pathologist works on a large number of digitized slides on a daily basis, opening the images to check whether such data are presented may slow down the workflow of the pathologist significantly. Therefore, the usability of the digital pathology platform needs to be improved.

### BRIEF DESCRIPTION OF THE DRAWINGS

The Detailed Description is set forth with reference to the accompanying figures. The use of the same reference numbers in different figures indicates similar or identical items. The detailed description is set forth with reference to the accompanying figures. In the figures, the left-most digit(s) of a reference number identifies the figure in which the reference number first appears. The use of the same reference numbers in different figures indicates similar or identical items or features. Furthermore, the drawings may be considered as providing an approximate depiction of the relative sizes of the individual applications within individual figures. However, the drawings are not to scale, and the relative sizes of the individual applications, both within individual figures and between the different figures, may vary from what is depicted. In particular, some of the figures may depict applications as a certain size or shape, while other figures may depict the same applications on a larger scale or differently shaped for the sake of clarity.

FIG. 1A and FIG. 1B illustrate an example slide panel which may be displayed on a user interface of a digital pathology platform according to implementations of this disclosure.

FIG. 1C illustrates an example slide panel including a settings button which may be displayed on a user interface of a digital pathology platform according to implementations of this disclosure.

FIG. 1D illustrates an example settings window which may be displayed on a user interface of a digital pathology platform according to implementations of this disclosure.

FIG. 1E illustrates an example slide panel which may be displayed in response to setting changes on a user interface of a digital pathology platform according to implementations of this disclosure.

FIG. 2A and FIG. 2B illustrate an example user interface of a digital pathology platform according to implementations of this disclosure.

FIG. 3A, FIG. 3B, and FIG. 3C illustrate an example user interface of a digital pathology platform according to implementations of this disclosure.

FIG. 4A, FIG. 4B, FIG. 4C, FIG. 4D, FIG. 4E, FIG. 4F, FIG. 4G, FIG. 4H, FIG. 4I, FIG. 4J, and FIG. 4K illustrate an example process for enhancing digital pathology platform according to implementations of this disclosure.

FIG. 5 illustrates an example apparatus for implementing the processes and methods described above.

FIG. 6 illustrates an example process for enhancing digital pathology platform according to implementations of this disclosure.

### DETAILED DESCRIPTION

This disclosure is generally directed to digital pathology. More particularly, this disclosure is directed to methods, apparatuses, and computer-readable media for enhancing digital pathology platform.

In various implementations, a user interface for a digital pathology platform may be displayed. A slide panel may be displayed on the user interface. A thumbnail of a digitized image of a specimen may be displayed, and one or more interactive icons associated with the slide panel may be displayed. The one or more interactive icons may indicate status associated with the slide panel.

The techniques described herein may improve the usability of the digital pathology platform.

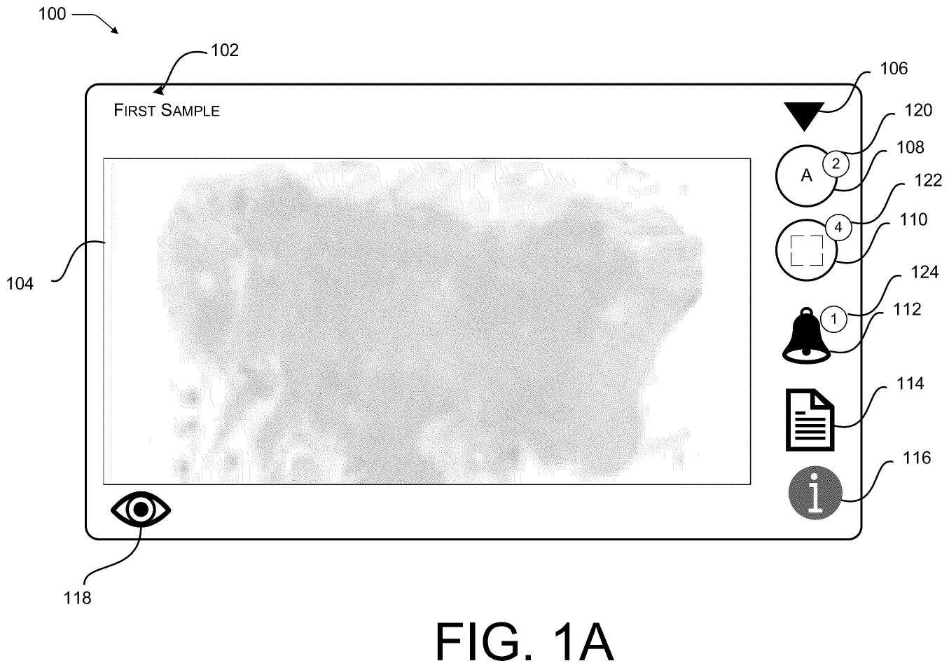

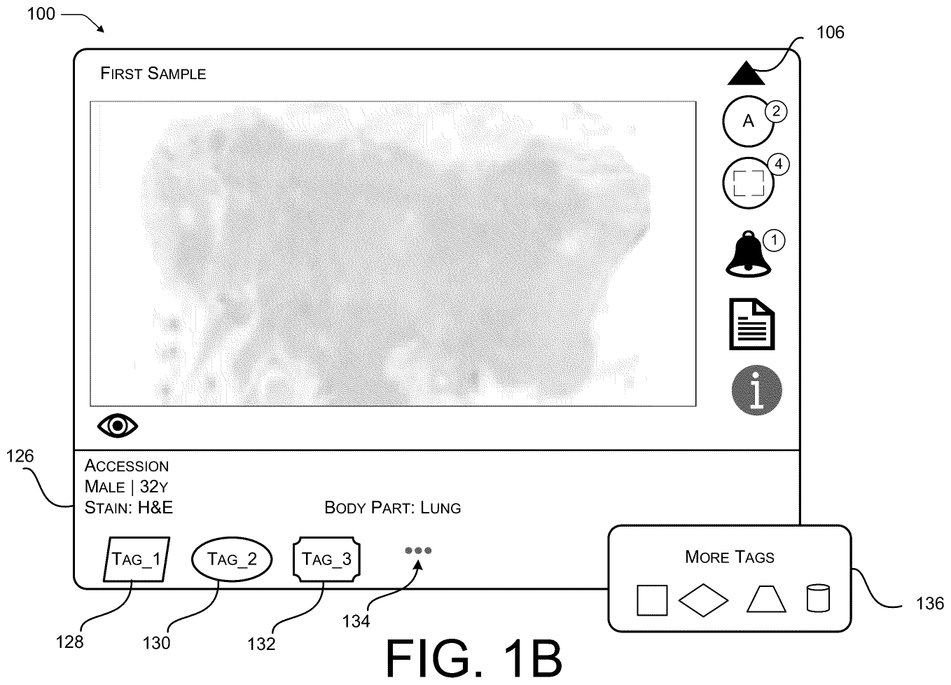

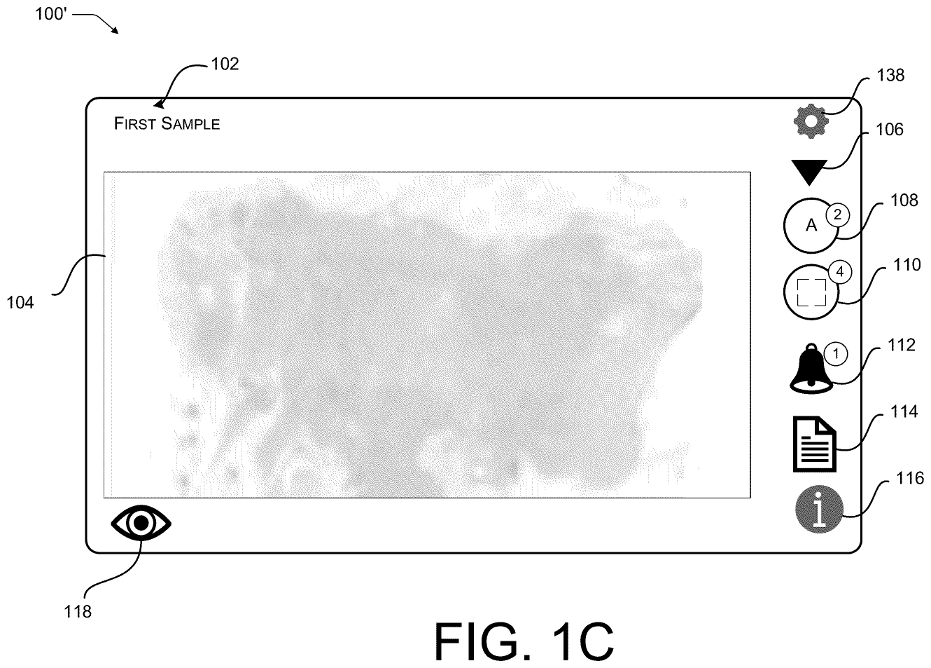

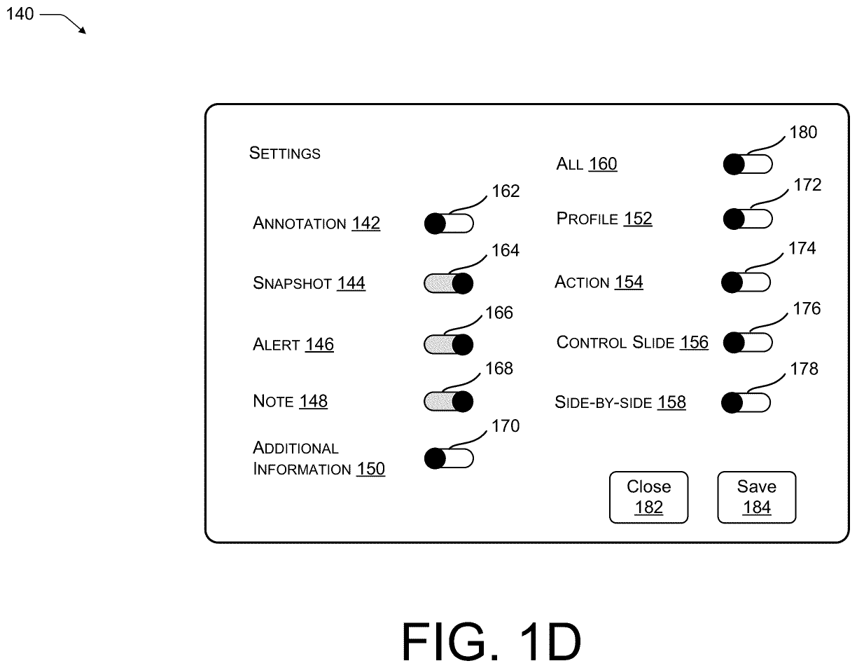

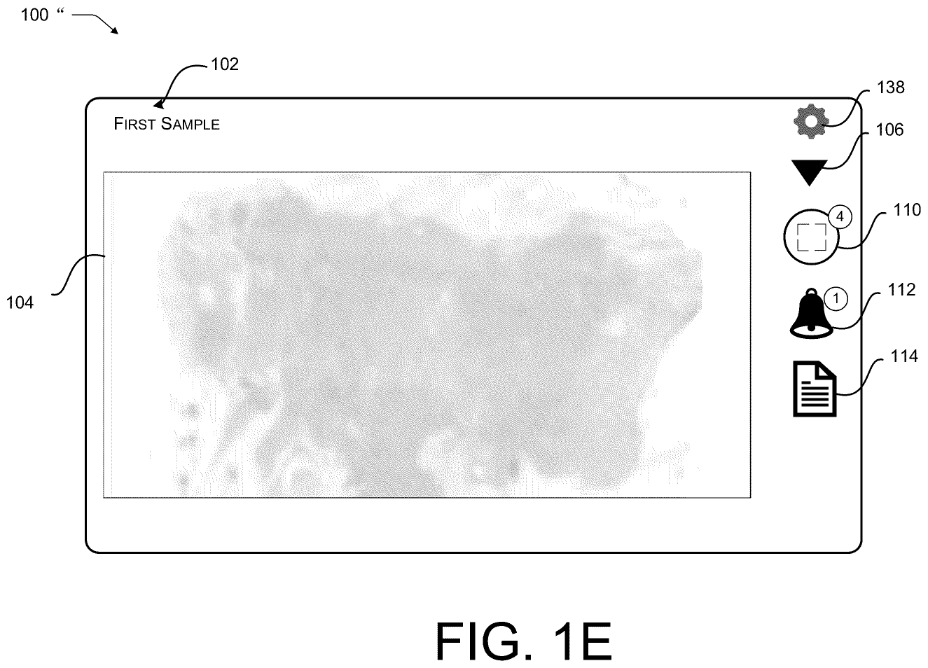

FIG. 1A and FIG. 1B illustrate an example slide panel 100 which may be displayed on a user interface of a digital pathology platform according to implementations of this disclosure.

Referring to FIG. 1A, the slide panel 100 may include an identifier 102, a thumbnail 104 of a digitized image of a specimen, and one or more interactive icons (including but not limited to 106, 108, 110, 112, 114, 116, and 118) associated with the thumbnail 104. The interactive icons are configured to indicate status associated with the slide panel 100. The status associated with the slide panel 100 includes, but is not limited to, whether the slide panel 100 is in a collapsed view mode or an expanded view mode, whether one or more annotations associated with one or more areas of interest of the digitized image of the specimen are available for further operations, whether one or more snapshots of one or more areas of interest of the digitized image of the specimen are available for further operations, whether one or more alerts associated with the digitized image of the specimen are available for further operations, whether a note associated with the digitized image of the specimen is available for further operations, whether additional information associated with the digitized image of the specimen is available for further operations, whether a follow-up action associated with the digitized image of the specimen is required, whether the digitized image of the specimen has been opened, etc.

In implementations, the identifier 102 may be configured to identify the name of the digitized image of the specimen.

In implementations, the thumbnail 104 may be configured to present the digitized image of the specimen in a reduced-size version.

Additionally, the thumbnail 104 may be further configured to be operable. For example, the user may operate the thumbnail 104 by clicking/tapping the thumbnail 104. In response to the operation on the thumbnail 104, the digitized image of the specimen may be displayed.

In implementations, the interactive icons may include a collapse-expand icon 106, an annotation icon 108, a snapshot icon 110, an alert icon 112, a note icon 114, an additional information icon 116, and an action icon 118. Additionally, or alternatively, other icons may be displayed with the slide panel 100. This disclosure is not limited thereto.

In implementations, the collapse-expand icon 106 may be configured to indicate whether the slide panel 100 is in a collapsed view mode or an expanded view mode. For example, the collapse-expand icon 106 may be an inverted triangle, indicating that the slide panel 100 is in the collapsed view mode. For example, the collapse-expand icon 106 may be a triangle, indicating that the slide panel 100 is in the expanded view mode. Though changing the shape of the collapse-expand icon 106 is described here as the manner of indicating whether the slide panel 100 is in a collapsed view mode or an expanded view mode, other manners may be used for the collapse-expand icon 106 to indicate whether the slide panel 100 is in a collapsed view mode or an expanded view mode, such as changing the color of the collapse-expand icon 106, changing the size of the collapse-expand icon 106, and so on. This disclosure is not limited thereto.

Additionally, the collapse-expand icon 106 may be further configured to be operable. For example, the user may operate the collapse-expand icon 106 by clicking/tapping the collapse-expand icon 106. In response to the operation, the slide panel 100 may be switched from the collapsed view mode to the expanded view mode when the slide panel 100 is in the collapsed view mode. Additionally, or alternatively, the slide panel 100 may be switched from the expanded view mode to the collapsed view mode when the slide panel 100 is in the expanded view mode.

In implementations, the annotation icon 108 may be configured to indicate whether one or more annotations associated with one or more areas of interest of the digitized image of the specimen are available for further operations. For example, the annotation icon 108 may be highlighted/unhighlighted to indicate that one or more annotations associated with one or more areas of interest of the digitized image of the specimen are available/unavailable for further operations. Though highlighting/unhighlighting the annotation icon 108 is described here as the manner of indicating that one or more annotations associated with one or more areas of interest of the digitized image of the specimen are available/unavailable for further operations, other manners may be used for the annotation icon 108 to indicate whether one or more annotations associated with one or more areas of interest of the digitized image of the specimen are available for further operations, such as changing the color of the annotation icon 108, changing the size of the annotation icon 108, changing the shape of the annotation icon 108, and so on. This disclosure is not limited thereto.

Additionally, the annotation icon 108 may be further configured to be operable. For example, the user may operate the annotation icon 108 by clicking/tapping the annotation icon 108. In response to the operation on the annotation icon 108, the one or more annotations associated with the one or more areas of interest of the digitized image of the specimen may be displayed. For example, an annotation associated with an area of interest of the digitized image of the specimen may be clinical comments added by a pathologist, such as “tumor”, “inflammatory cells”, etc.

Additionally, the annotation icon 108 may include an annotation tag 120 which may be configured to indicate a number of the annotations. For example, the annotation tag 120 may show the number “2”, indicating that there are two annotations associated with one or more areas of interest of the digitized image of the specimen are available for further operations. In implementations, multiple annotations may be associated with the same area of interest or different areas of interest of the digitized image of the specimen. For example, multiple annotations associated with the same area of interest may be added by different pathologists. For example, multiple annotations associated with the same area of interest may be added at different times. This disclosure is not limited thereto.

In implementations, the snapshot icon 110 may be configured to indicate whether one or more snapshots of one or more areas of interest of the digitized image of the specimen are available for further operations. For example, the snapshot icon 110 may be highlighted/unhighlighted to indicate that one or more snapshots associated with one or more areas of interest of the digitized image of the specimen are available/unavailable for further operations. Though highlighting/unhighlighting the snapshot icon 110 is described here as the manner of indicating that one or more snapshots associated with one or more areas of interest of the digitized image of the specimen are available/unavailable for further operations, other manners may be used for the snapshot icon 110 to indicate whether one or more snapshots associated with one or more areas of interest of the digitized image of the specimen are available for further operations such as changing the color of the snapshot icon 110, changing the size of the snapshot icon 110, changing the shape of the snapshot icon 110, and so on. This disclosure is not limited thereto.

Additionally, the snapshot icon 110 may be further configured to be operable. For example, the user may operate the snapshot icon 110 by clicking/tapping the snapshot icon 110. In response to the operation on the snapshot icon 110, the one or more snapshots associated with the one or more areas of interest of the digitized image of the specimen may be displayed.

Additionally, the snapshot icon 110 may include a snapshot tag 122 which may be configured to indicate a number of the snapshots. For example, the snapshot tag 122 may show the number “4”, indicating that there are four snapshots associated with one or more areas of interest of the digitized image of the specimen that are available for further operations. Multiple snapshots may be associated with the same area of interest or different areas of interest of the digitized image of the specimen. For example, multiple snapshots may be taken for the same area of interest with different magnifications such as 40×, 100×, and so on. For example, multiple snapshots may be taken for the same area of interest with different definitions. This disclosure is not limited thereto.

In implementations, the alert icon 112 may be configured to indicate whether one or more alerts associated with the digitized image of the specimen are available for further operations. For example, the alert icon 112 may be highlighted/unhighlighted to indicate that one or more alerts associated with the digitized image of the specimen are available/unavailable for further operations. Though highlighting/unhighlighting the alert icon 112 is described here as the manner of indicating that one or more alerts associated with the digitized image of the specimen are available/unavailable for further operations, other manners may be used for the alert icon 112 to indicate whether one or more alerts associated with the digitized image of the specimen is available for further operations, such as changing the color of the alert icon 112, changing the size of the alert icon 112, changing the shape of the alert icon 112, and so on. This disclosure is not limited thereto.

Additionally, the alert icon 112 may be further configured to be operable. For example, the user may operate the alert icon 112 by clicking/tapping the alert icon 112. In response to the operation on the alert icon 112, the one or more alerts associated with the digitized image of the specimen may be displayed. Additionally, the user may manage the one or more alerts. For example, the user may subscribe to an alert which notifies that the first sample is reviewed by a certain pathologist. In that case, when the first sample is reviewed by the certain pathologist, the alert icon 112 associated with the first sample may be highlighted, indicating that an alert is available for further operation. The user may click the alert icon 112, and the content of the alert may be displayed, indicating that the first sample is reviewed by the certain pathologist.

Additionally, the alert icon 112 may include an alert tag 124 which may be configured to indicate a number of the alerts. For example, the alert tag 124 may show the number “1”, indicating that there is one alert associated with the digitized image of the specimen that is available for further operations.

In implementations, the note icon 114 may be configured to indicate whether a note associated with the digitized image of the specimen is available for further operations. For example, the note icon 114 may be highlighted/unhighlighted to indicate that a note associated with the digitized image of the specimen is available/unavailable for further operations. Though highlighting/unhighlighting the note icon 114 is described here as the manner of indicating that a note associated with the digitized image of the specimen is available/unavailable for further operations, other manners may be used for the note icon 114 to indicate whether a note associated with the digitized image of the specimen is available for further operations, such as changing the color of the note icon 114, changing the size of the note icon 114, changing the shape of the note icon 114, and so on. This disclosure is not limited thereto.

Additionally, the note icon 114 may be further configured to be operable. For example, the user may operate the note icon 114 by clicking/tapping the note icon 114. In response to the operation on the note icon 114, the note associated with the digitized image of the specimen may be displayed.

In implementations, the additional information icon 116 may be configured to indicate whether additional information associated with the digitized image of the specimen is available for further operations. For example, the additional information icon 116 may be highlighted/unhighlighted to indicate that additional information associated with the digitized image of the specimen is available/unavailable for further operations. Though highlighting/unhighlighting the additional information icon 116 is described here as the manner of indicating that additional information associated with the digitized image of the specimen is available/unavailable for further operations, other manners may be used for the additional information icon 116 to indicate whether additional information associated with the digitized image of the specimen is available for further operations, such as changing the color of the additional information icon 116, changing the size of the additional information icon 116, changing the shape of the additional information icon 116, and so on. This disclosure is not limited thereto.

Additionally, the additional information icon 116 may be further configured to be operable. For example, the user may operate the additional information icon 116 by clicking/tapping the additional information icon 116. In response to the operation on the additional information icon 116, the additional information may be displayed. For example, the additional information may include, but is not limited to, the image type of the digitized image of the specimen (such as Portable Network Graphics (PNG), Joint Photographic Expert Group (JPEG), and so on), the image size and resolution, the brand/model/type of the scanner which scans the specimen to generate the digitized image, the scan magnification, etc.

In implementations, the action icon 118 may be configured to indicate whether a follow-up action associated with the digitized image of the specimen is required. For example, the action icon 118 may be highlighted/unhighlighted to indicate that a follow-up action associated with the digitized image of the specimen is required/unrequired. Though highlighting/unhighlighting the action icon 118 is described here as the manner of indicating that a follow-up action associated with the digitized image of the specimen is required/unrequired, other manners may be used for the action icon 118 to indicate whether a follow-up action associated with the digitized image of the specimen is required, such as changing the color of the action icon 118, changing the size of the action icon 118, changing the shape of the action icon 118, and so on. This disclosure is not limited thereto. In implementations, the follow-up action may include reviewing by another pathologist. For example, the user may require another pathologist to review the first sample.

Additionally, action icon 118 may be further configured to be operable. For example, the user may operate the action icon 118 by clicking/tapping the action icon 118. In response to the operation on the action icon 118, the slide panel 100 may be switched from a normal state to a hidden state when the slide panel 100 is in the normal state. Additionally, or alternatively, in response to the operation on the action icon 118, the slide panel 100 may be switched from the hidden state to the normal state when the slide panel 100 is in the hidden state.

In the normal state, the slide panel 100 may be displayed without additional visual effects. In the hidden state, the slide panel 100 may be displayed without additional visual effects, such as blur, shadow, and so on. This disclosure is not limited thereto. The state change of the slide panel 100 provides convenience for the user. The user may see whether the slide panel 100 requires follow-up action from the appearance of the slide panel 100 directly without opening the digital image to check further data, and the usability of the digital pathology platform is improved.

Referring to FIG. 1B, the collapse-expand icon 106 is in a triangle shape, indicating that the slide panel 100 is in the expanded view mode. The expanded part 126 of the slide panel 100 may be shown in the expanded view mode. For example, the expanded part 126 of the slide panel 100 may show information such as the source of the specimen, the stain type of the specimen, the body part of the specimen, and tags (such as tag_1 128, Tag_2 130, and tag_3 132) associated with the specimen.

In implementation, the tags associated with the specimen may be configured to be an index to indicate more information associated with the specimen. For example, various shapes of the tags may indicate various categorizations of the specimen. Additionally, more information associated with the specimen may be indicated through other manners such as various colors, various greyscales, and so on of the tags. This disclosure is not limited thereto.

In implementations, the expanded part 126 of the slide panel 100 may further include a more tags icon 134. The more tags icon 134 may be configured to be operable. For example, the user may operate the more tags icon 134 by clicking/tapping the more tags icon 134. In response to the operation on the more tags icon 134, a pop-up window 136 may be displayed. The pop-up window 136 may show more tags for the user to select.

With the slide panel 100, the one or more interactive icons enable the user to know the status associated with the slide panel 100 without opening the digitized image of the specimen. Additionally, the one or more interactive icons are operable such that the user may view the data associated with the slide panel such as annotations, snapshots, alerts, notes, additional information, follow-up action requirements, and so on by operating the one or more interactive icons without opening the digitized image of the specimen. The usability of the digital pathology platform may be improved. The workflow of the user may be facilitated.

FIG. 1C illustrates an example slide panel 100′ including a settings button which may be displayed on a user interface of a digital pathology platform according to implementations of this disclosure.

Referring to FIG. 1C, the slide panel 100′ may include an identifier 102, a thumbnail 104 of a digitized image of a specimen, and one or more interactive icons (including but not limited to 106, 108, 110, 112, 114, 116, and 118) associated with the thumbnail 104. The interactive icons can be the same as those in FIGS. 1A and 1B. The difference between FIG. 1C and FIG. 1A is that FIG. 1C further includes a settings button 138 (which can be an interactive button).

FIG. 1D illustrates an example settings window 140 which may be displayed on a user interface of a digital pathology platform according to implementations of this disclosure.

In implementations, the settings button 138 may be configured to be operable. For example, the user may operate the settings button 138 by clicking/tapping the settings button 138. In response to the operation on the settings button 138, a user interface of a digital pathology platform may display a settings window 140. The user may customize the settings of the slide panel 100′ via the settings window 140.

In implementations, the settings window 140 may display one or more operable fields configured to change the settings of the slide panel 100′. The operable fields may include, but are not limited to, an annotation field 142, a snapshot field 144, an alert field 146, a note field 148, an additional information field 150, a profile field 152, an action field 154, a control slide field 156, and a side-by-side field 158. Note that these operable fields are exemplary rather than limiting. Some of these operational fields can be omitted or replaced. Additional operable fields can be added to the settings window 140 based on actual needs.

In implementations, an individual operable field can be configured to be operable to set a feature (or an interactive icon) associated with the slide panel 100′. For example, the annotation field 142 can be configured to be operable to set the annotation icon 108. The snapshot field 144 can be configured to be operable to set the snapshot icon 110. The alert field 146 can be configured to be operable to set the alert icon 112. The note field 148 can be configured to be operable to set the note icon 114. The additional information field 150 can be configured to be operable to set the note icon 116. The profile field 152 can be configured to be operable to set the profile of the slide panel 100′. The action field 154 can be configured to be operable to set the action icon 118.

The settings window 140 may further include an all field 160 which can be configured to be operable to set all the operable fields together.

In some examples, an individual operable field can include an operable trigger such as a slider, a button, a checkbox, a drop list, any combination thereof, or the like. For example, the annotation field 142 can include a first slider 162. When the first slider 162 is turned on (e.g., when the first slider 162 is on the right side), the annotation icon 108 is displayed along with the thumbnail 104 in the slide panel 100′. When the first slider 162 is turned off (e.g., when the first slider 162 is on the left side), the annotation icon 108 is not displayed or hidden.

For example, the snapshot field 144 can include a second slider 164. When the second slider 164 is turned on (e.g., when the second slider 164 is on the right side), the snapshot icon 110 is displayed along with the thumbnail 104 in the slide panel 100′. When the second slider 164 is turned off (e.g., when the second slider 164 is on the left side), the snapshot icon 110 is not displayed or hidden.

For example, the alert field 146 can include a third slider 166. When the third slider 166 is turned on (e.g., when the third slider 166 is on the right side), the alert icon 112 is displayed along with the thumbnail 104 in the slide panel 100′. When the third slider 166 is turned off (e.g., when the third slider 166 is on the left side), the alert icon 112 is not displayed or hidden.

For example, the note field 148 can include a fourth slider 168. When the fourth slider 168 is turned on (e.g., when the fourth slider 168 is on the right side), the note icon 114 is displayed along with the thumbnail 104 in the slide panel 100′. When the fourth slider 168 is turned off (e.g., when the fourth slider 168 is on the left side), the note icon 114 is not displayed or hidden.

For example, the additional information field 150 can include a fifth slider 170. When the fifth slider 170 is turned on (e.g., when the fifth slider 170 is on the right side), the additional information icon 114 is displayed along with the thumbnail 104 in the slide panel 100′. When the fifth slider 170 is turned off (e.g., when the fifth slider 170 is on the left side), the additional information icon 114 is not displayed or hidden.

For example, the profile field 152 can include a sixth slider 172. When the sixth slider 172 is turned on (e.g., when the sixth slider 172 is on the right side), the profile information the slide panel 100′ is displayed. When the sixth slider 172 is turned off (e.g., when the sixth slider 172 is on the left side), the profile information is not displayed or hidden.

For example, the action field 154 can include a seventh slider 174. When the seventh slider 174 is turned on (e.g., when the seventh slider 174 is on the right side), the action icon 118 is displayed along with the thumbnail 104 in the slide panel 100′. When the seventh slider 174 is turned off (e.g., when the seventh slider 174 is on the left side), the action icon 118 is not displayed or hidden.

For example, the control slide field 156 can include an eighth slider 176. When the eighth slider 176 is turned on (e.g., when the eighth slider 176 is on the right side), a digital image of a control slide is displayed along with the thumbnail 104 in the slide panel 100′. When the eighth slider 176 is turned off (e.g., when the eighth slider 176 is on the left side), the digital image of the control slide is not displayed or hidden. In some examples, a control slide, also known as a positive control slide, can be a specimen slide that is intentionally prepared to contain tissue or cells known to exhibit specific characteristics, reactions (such as inflammation reactions or the like), diseases (such as tumors or the like). In implementations, displaying a digital image of a control slide along with the thumbnail 104 can help the user (such as a pathologist) to evaluate the staining quality of the specimen, verify the accuracy of the staining reactions, and determine whether the diagnostic or research results are valid. The control slides can serve as references against which the unknown specimens can be compared, helping to ensure the accuracy and reliability of diagnostic results. The control slides can ensure the and reproducibility of laboratory procedures, which is useful for efficient diagnosis and research findings. In implementations, the digital image of the control slide can be displayed together with the thumbnail 104 in various manners, such as in a side-by-side manner, in a picture-in-picture manner, in an overlaying manner, in a vertical arrangement, or the like.

For example, the side-by-side field 158 can include a ninth slider 178. When the ninth slider 178 is turned on (e.g., when the ninth slider 178 is on the right side), the digital image of the control slide can be displayed side-by-side with the thumbnail 104 in the slide panel 100′. When the ninth slider 178 is turned off (e.g., when the ninth slider 178 is on the left side), the digital image of the control slide is not displayed side-by-side with the thumbnail 104.

For example, the all field 160 can include a tenth slider 180. When the tenth slider 180 is turned on (e.g., when the tenth slider 180 is on the right side), the first to the ninth sliders are all turned on. When the tenth slider 180 is turned off (e.g., when the tenth slider 180 is on the left side), the first to the ninth sliders can be individually configured.

Note that in FIG. 1D, sliders (e.g., 162-180) are shown as examples rather than limitations. The operable fields 142-160 may include other operable triggers such as a slider, a button, a checkbox, a drop list, any combination thereof, or the like. In implementations, a user can configure the operable fields 142-160 by operating the sliders (e.g., 162-180). An individual operable field (e.g., 142-160) can receive an operation. In response to the operation, the individual operable field (e.g., 142-160) can change the status of the slider (e.g., from a turned on status to a turned off status, or from a turned off status to a turned on status).

In implementations, the settings window 140 may further include a close button 182 and a save button 184. The close button 182 can receive an operation. In response to the operation, the settings window 140 can be closed. The save button 184 can receive an operation. In response to the operation, the configurations of the operable field (e.g., 142-160) can be saved.

FIG. 1E illustrates an example slide panel 100″ which may be displayed in response to setting changes on a user interface of a digital pathology platform according to implementations of this disclosure.

Referring to FIG. 1E, the slide panel 100′ may include an identifier 102, a thumbnail 104 of a digitized image of a specimen, and one or more interactive icons associated with the thumbnail 104. In this example, the slide panel 100′ is displayed based on the settings configured in the settings window 140 shown in FIG. 1D. In FIG. 1E, the snapshot icon 110, the alert icon 112, and the note icon 114 are displayed. Because in the settings window 140, the snapshot field 144, the alert field 146, and the note field 148 are turned on. On the other hand, the annotation icon 108, the additional information icon 116, and the action icon 118 are not displayed. Because the annotation field 14, the additional information field 150, and the action field 154 are turned off.

As such, the user can conveniently customize the display manner of the slide panel, selecting which icons/information to be displayed/hidden, streamlining the workflow, and ensuring efficient decision-making.

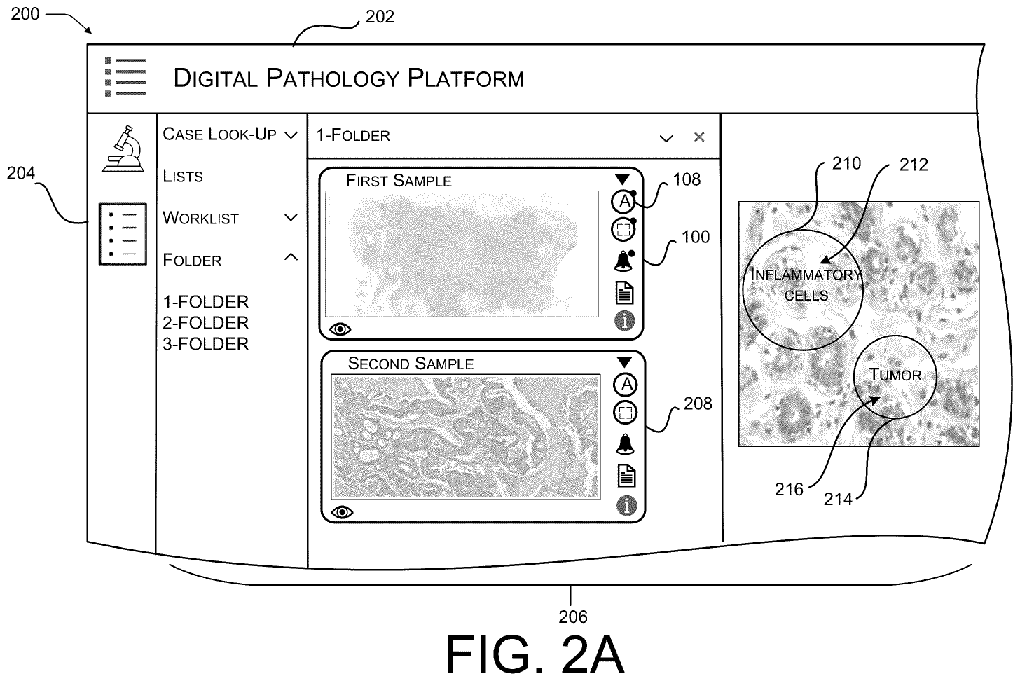

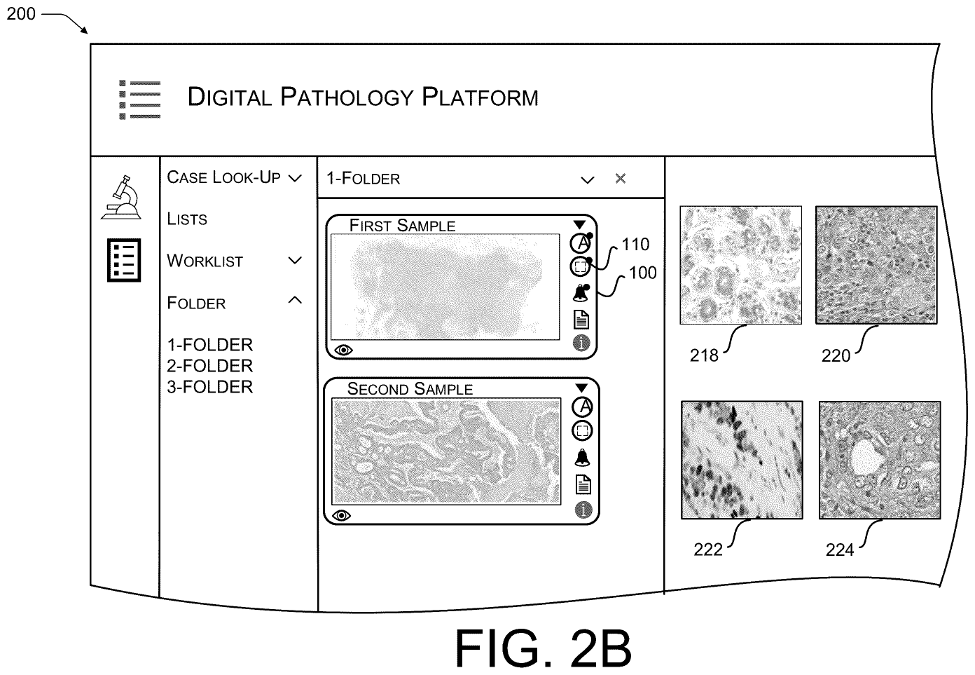

FIG. 2A and FIG. 2B illustrate an example user interface 200 of a digital pathology platform according to implementations of this disclosure.

Referring to FIG. 2A, the user interface 200 may include a header 202, a sidebar 204, and a main display area 206.

In implementations, the header 202 may be configured to show the name of the digital pathology platform.

In implementations, the sidebar 204 may be configured to show buttons of applications (Apps). A button is an interactive element on the user interface 200 that enables the user to directly communicate with the user interface. An application is a type of software that allows the user to perform tasks and may be integrated with the digital pathology platform.

In implementations, the main display area 206 may be configured to display texts, graphics, images, directories, background details, etc. This disclosure is not limited thereto.

For example, the main display area 206 may display slide panel “first sample” 100 and slide panel “second sample” 208.

As an example, the user may operate the annotation icon 108 by clicking/tapping the annotation icon 108. In response to the operation on the annotation icon 108, a first area of interest 210 and a first annotation 212 associated with the first area of interest 210, and a second area of interest 214 and a second annotation 216 associated with the second area of interest 214 may be displayed in the main area 206 of the user interface 200. Other numbers of areas of interest and associated annotations may be shown. Multiple annotations may be associated with the same area of interest. For example, annotations associated with areas of interest of the digitized image of the specimen may be clinical comments added by a pathologist, such as “tumor”, “inflammatory cells”, etc. This disclosure is not limited thereto. Additional details of the annotations are described throughout this disclosure and are not repeated here.

Referring to FIG. 2B, as an example, the user may operate the snapshot icon 110 by clicking/tapping the snapshot icon 110. In response to the operation on the snapshot icon 110, a first snapshot 218, a second snapshot 220, a third snapshot 222, and a fourth snapshot 224 may be displayed in the main area 206 of the user interface 200. Other numbers of snapshots may be shown. Multiple snapshots may be associated with the same area of interest or different areas of interest. For example, multiple snapshots may be taken for the same area of interest with different magnifications such as 40×, 100×, and so on. This disclosure is not limited thereto. Additional details of the snapshots are described throughout this disclosure and are not repeated here.

With the user interface 200, the one or more interactive icons enable the user to view the associated data such as annotations, snapshots, and so on by operating the one or more interactive icons without opening the digitized image of the specimen. The usability of the digital pathology platform may be improved. The workflow of the user may be facilitated.

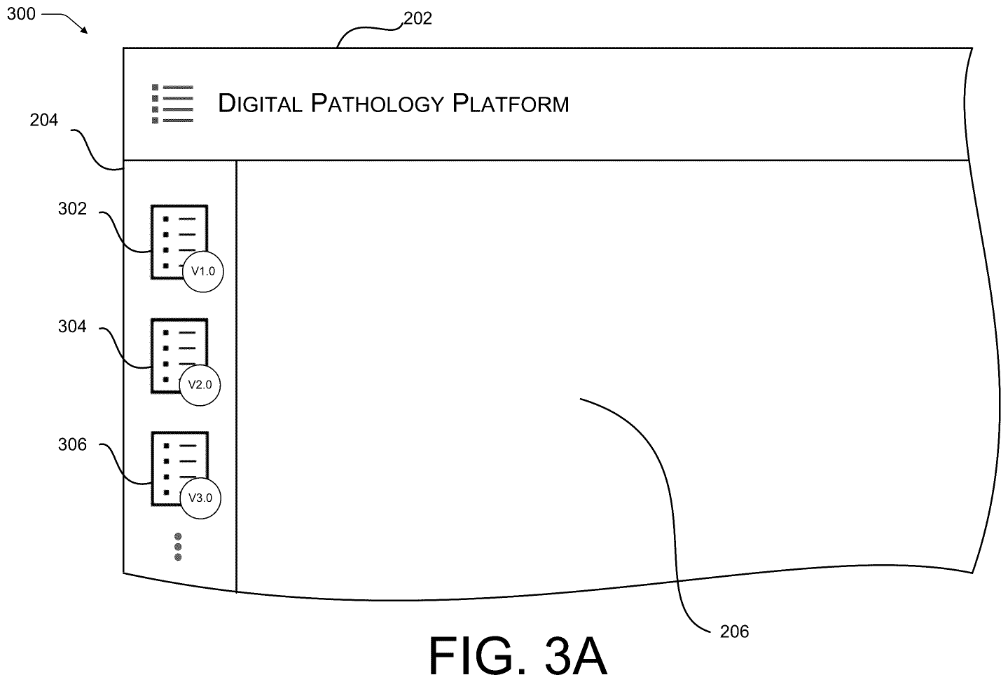

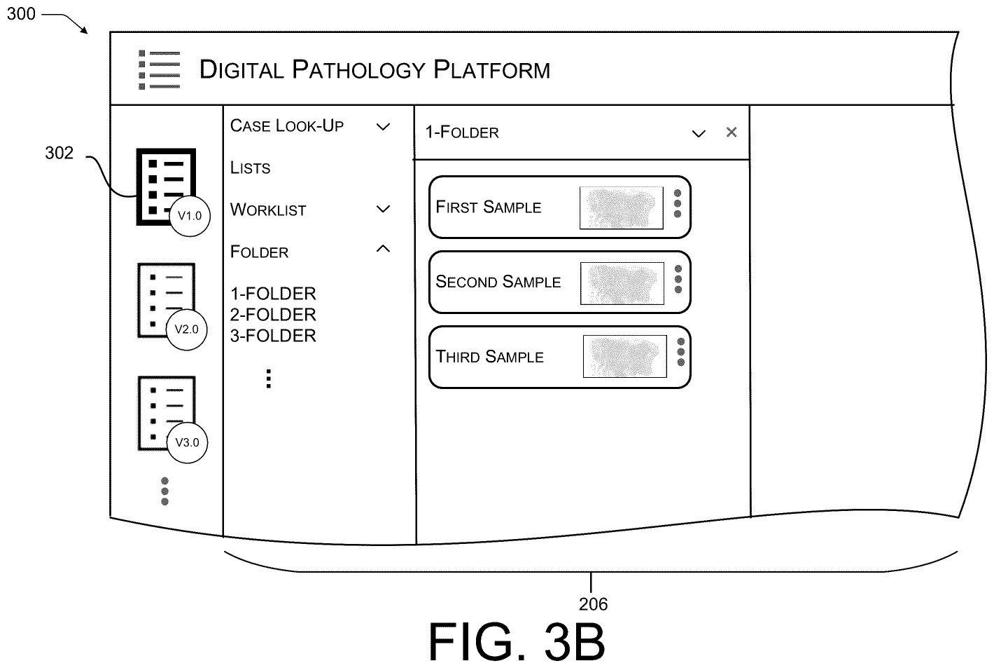

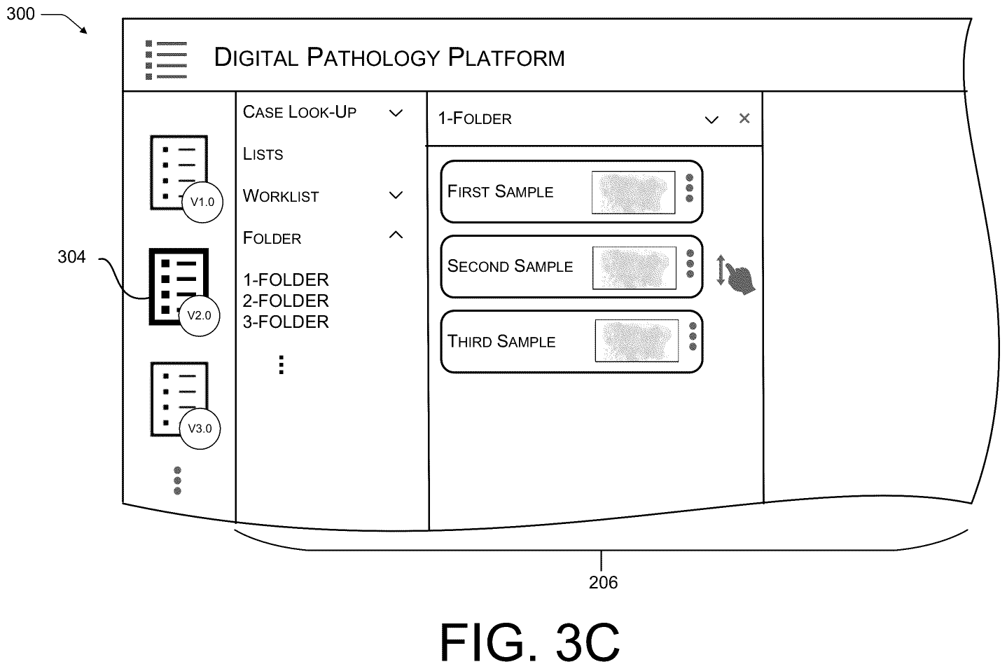

FIG. 3A, FIG. 3B, and FIG. 3C illustrates an example user interface 300 of a digital pathology platform according to implementations of this disclosure.

Referring to FIG. 3A, the user interface 300 may have the same arrangement as the user interface 200. The user interface 300 may include a header 202, a sidebar 204, and a main display area 206.

In implementations, the sidebar 204 may include a first button 302 of a first version of an application, a second button 304 of a second version of the application, a third button 306 of a third version of the application, etc.

Referring to FIG. 3B, the user may operate the first button 302 by clicking/tapping the first button 302. In response to the operation on the first button 302, the first version of the application may be displayed in the main display area 206.

Referring to FIG. 3C, the user may operate the second button 304 by clicking/tapping the second button 304. In response to the operation on the second button 304, the second version of the application may be displayed in the main display area 206.

In implementations, the second version of the application may include improved features that are not available in the first version of the application. For example, with the second version of the application, the user may change the order of slide panels by dragging and dropping the slide panels. On the other hand, the order of slide panels may not be changed in the first version of the application.

With the user interface 300, various versions of the same application may be accessible to the user at the same time. In practice, when a new version of an application is released, the user may need to be trained for several weeks to learn how to use the new version of the application. During the training period, the user may use the old version of the application to work. Additionally, the user may need to use different versions of the application for other reasons. This disclosure is not limited thereto. Thus, the usability of the digital pathology platform is improved. The workflow of the user may be facilitated.

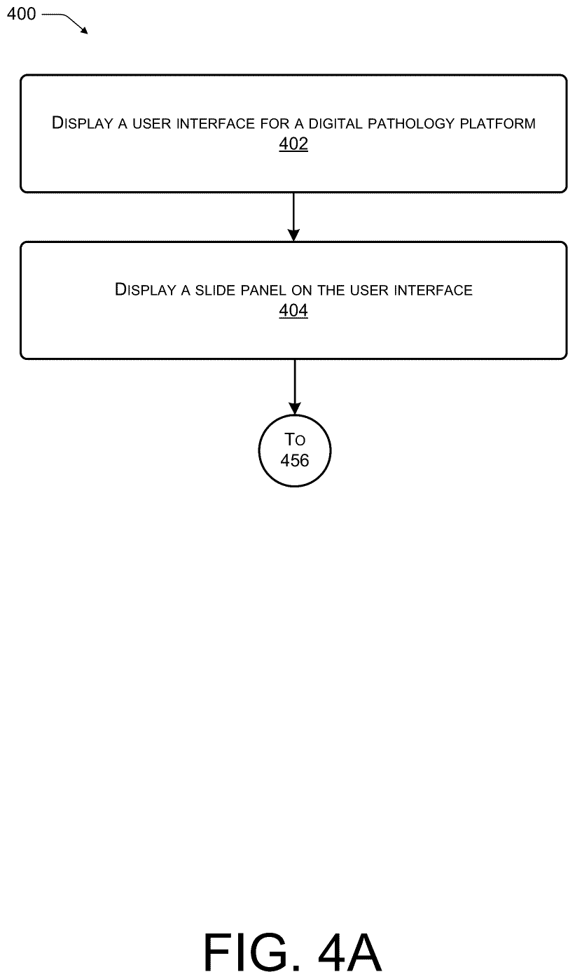

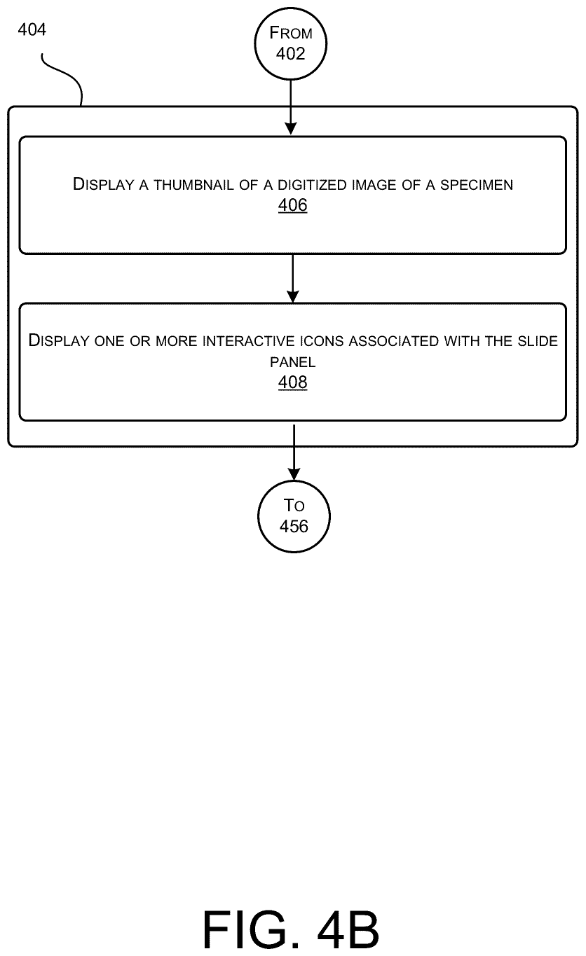

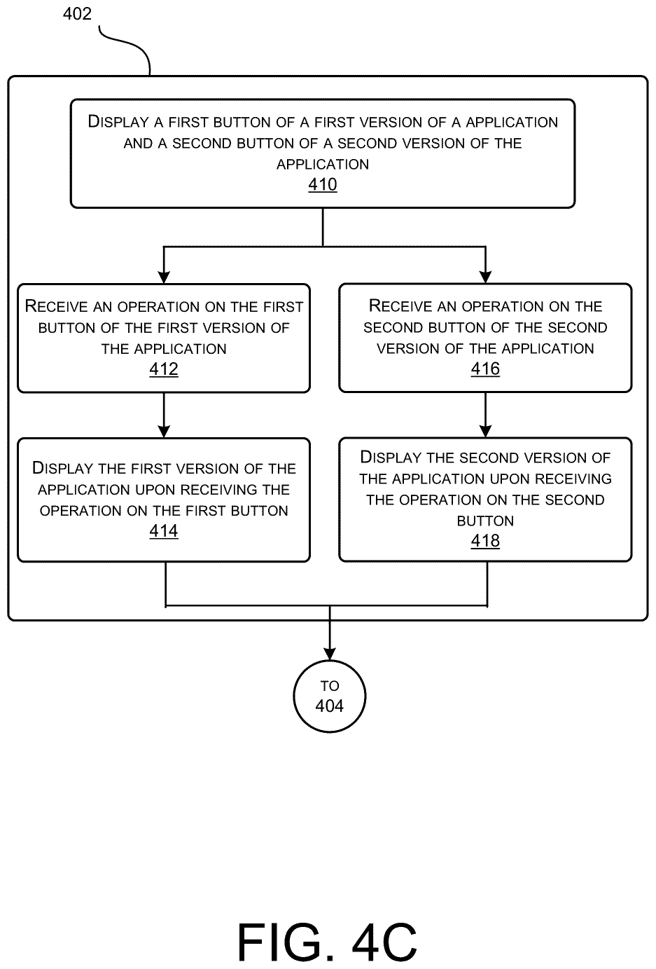

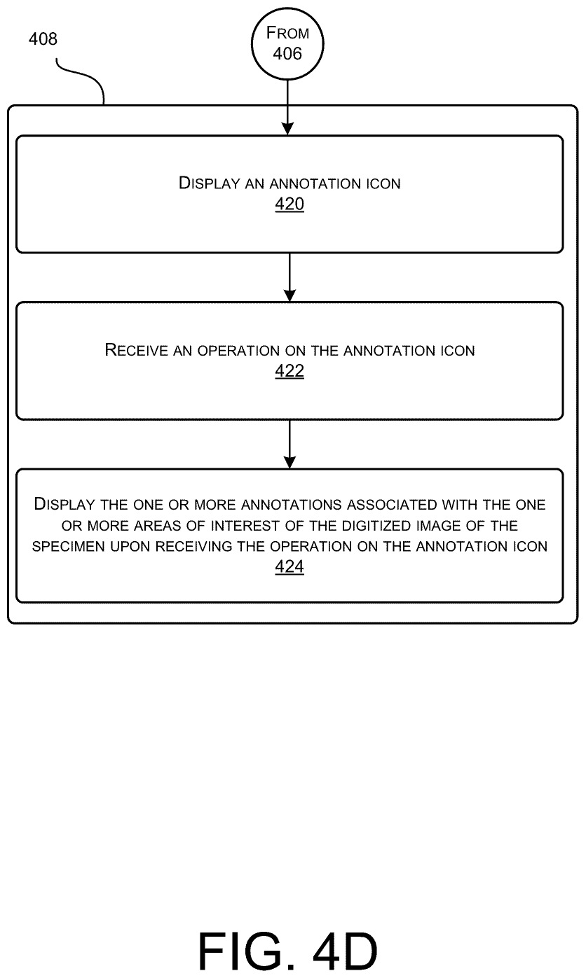

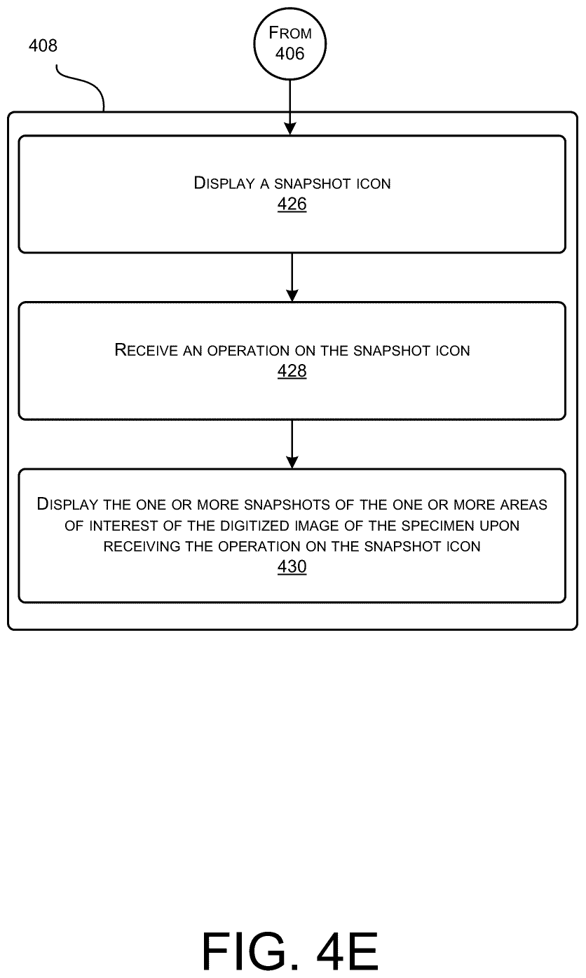

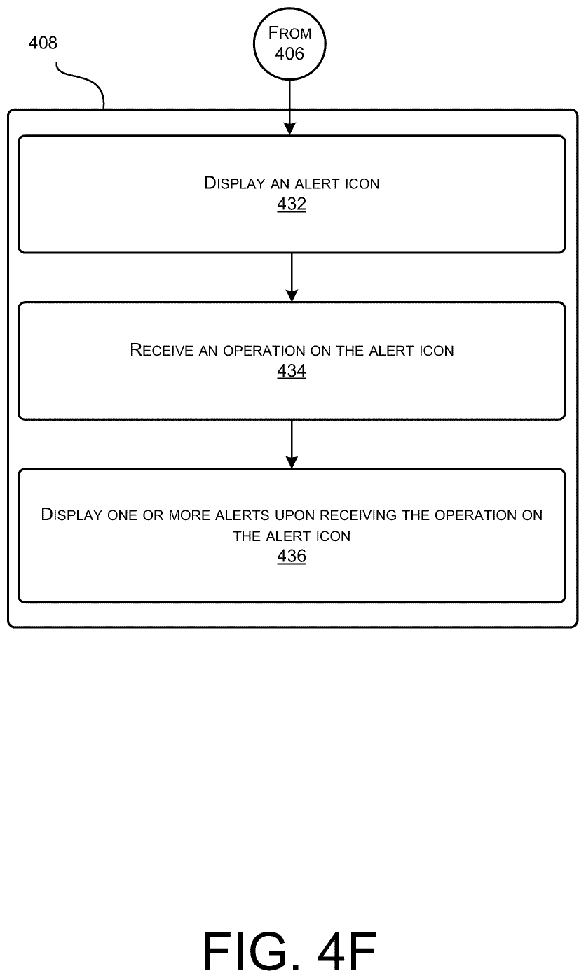

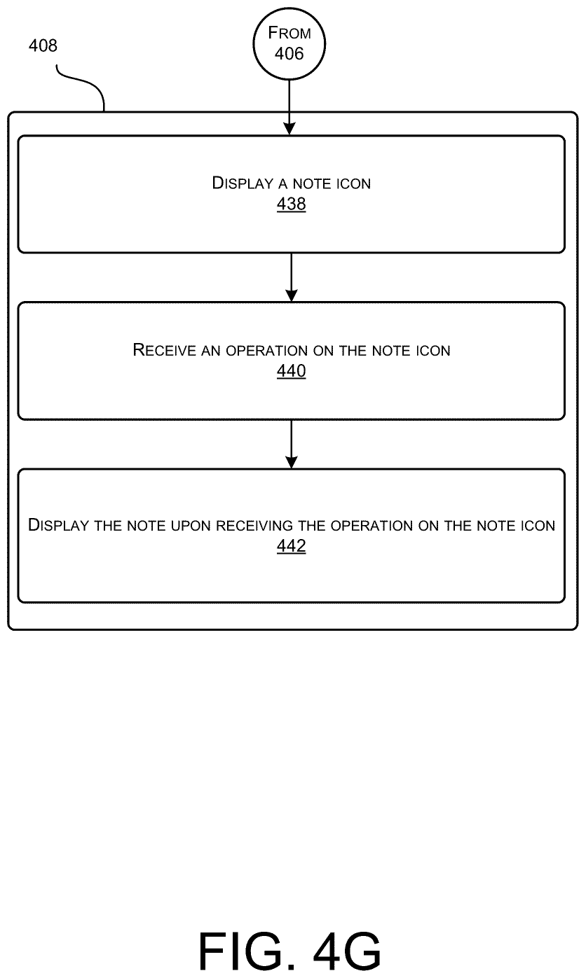

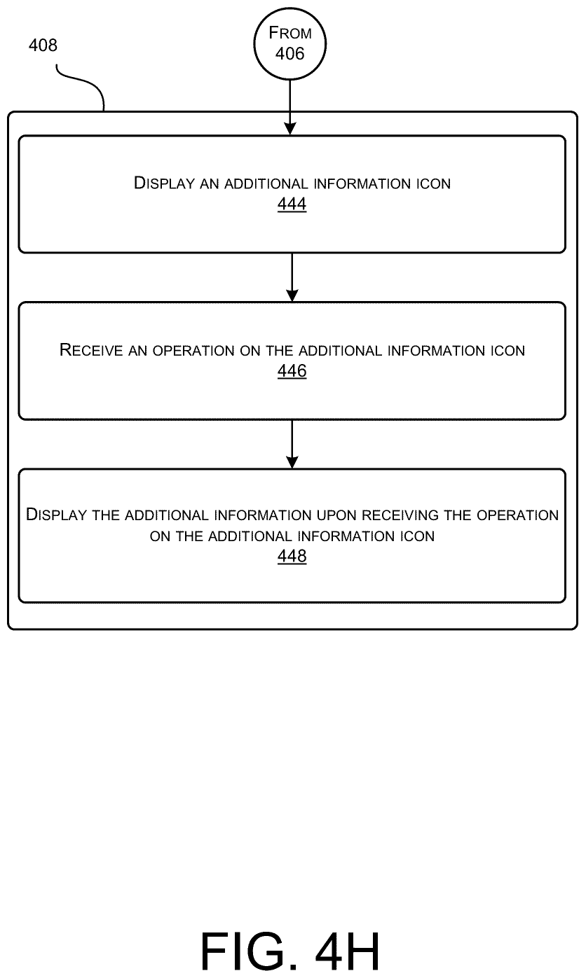

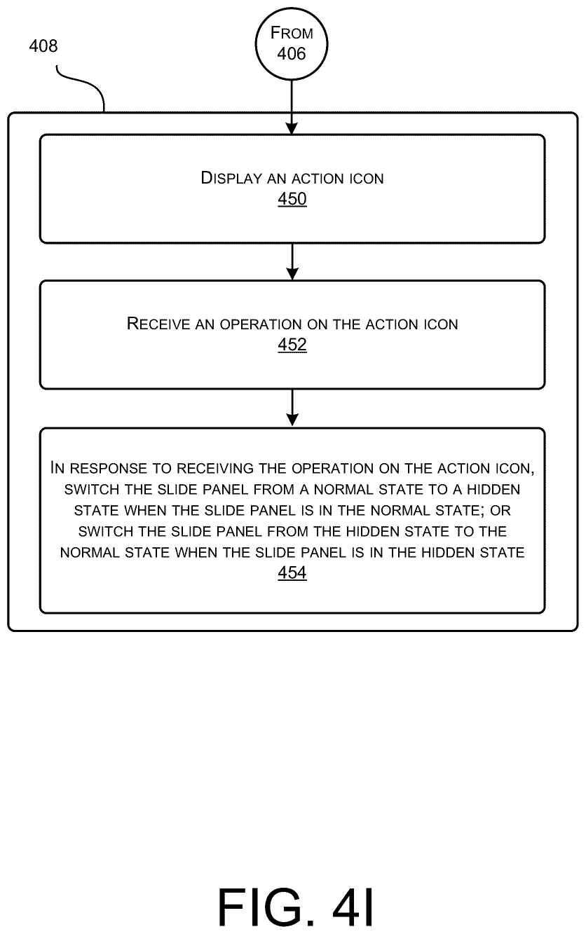

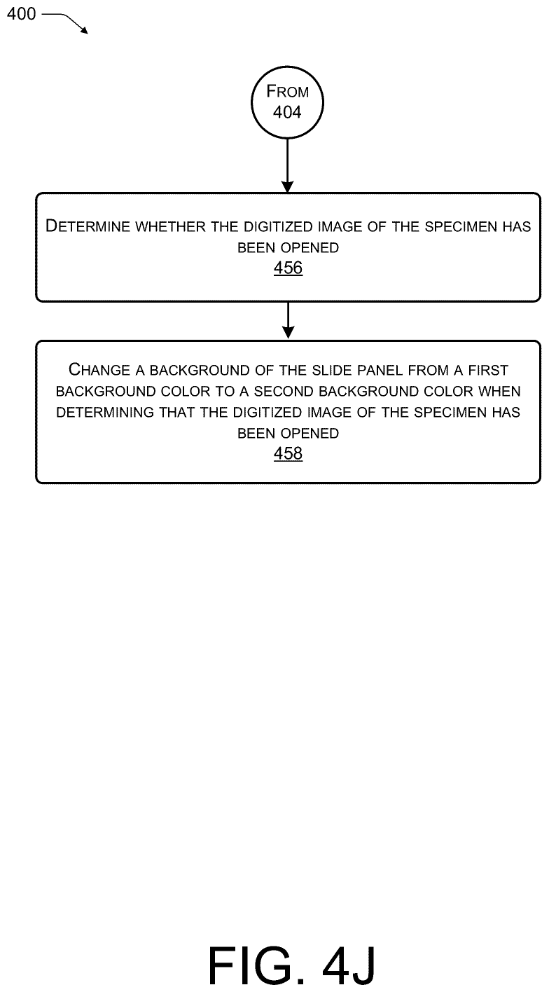

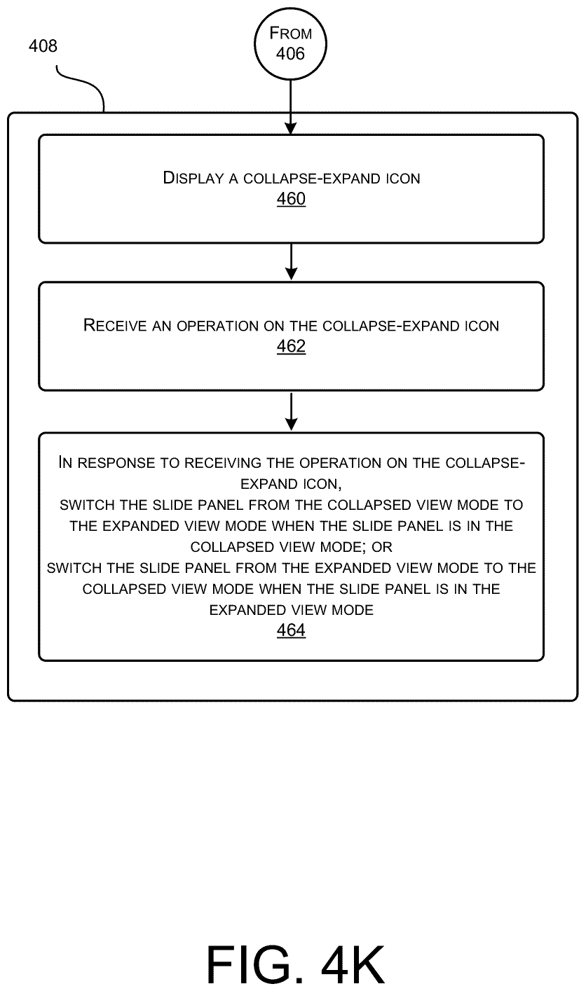

FIG. 4A, FIG. 4B, FIG. 4C, FIG. 4D, FIG. 4E, FIG. 4F, FIG. 4G, FIG. 4H, FIG. 4I, FIG. 4J, and FIG. 4K illustrate an example process 400 for enhancing digital pathology platform according to implementations of this disclosure.

Referring to FIG. 4A, process 400 may include the following.

At block 402, operations may include displaying a user interface for a digital pathology platform.

At block 404, operations may include displaying a slide panel on the user interface. Additional details of the slide panel are described throughout the disclosure and are not repeated here.

Referring to FIG. 4B, block 404 may include the following.

At block 406, operations may include displaying a thumbnail of a digitized image of a specimen. Additional details of the thumbnail are described throughout the disclosure and are not repeated here.

At block 408, operations may include displaying one or more interactive icons associated with the slide panel. The one or more interactive icons indicating status associated with the slide panel.

Referring to FIG. 4C, block 402 may include the following.

At block 410, operations may include displaying a first button of a first version of an application and a second button of a second version of the application.

At block 412, operations may include receiving an operation on the first button of the first version of the application.

At block 414, operations may include displaying the first version of the application upon receiving the operation on the first button.

At block 416, operations may include receiving an operation on the second button of the second version of the application.

At block 418, operations may include displaying the second version of the application upon receiving the operation on the second button.

Additional details of the version switching of the application are described throughout the disclosure and are not repeated here.

Referring to FIG. 4D, block 408 may include the following.

At block 420, operations may include displaying an annotation icon. The annotation icon may indicate whether one or more annotations associated with one or more areas of interest of the digitized image of the specimen are available for further operations. Additionally, displaying the annotation icon includes displaying an annotation tag, the annotation tag indicating a number of the one or more annotations. Additional details of the annotation icon are described throughout the disclosure and are not repeated here.

At block 422, operations may include receiving an operation on the annotation icon.

At block 424, operations may include displaying the one or more annotations associated with the one or more areas of interest of the digitized image of the specimen upon receiving the operation on the annotation icon.

Referring to FIG. 4E, block 408 may include the following.

At block 426, operations may include displaying a snapshot icon. The snapshot icon may indicate whether one or more snapshots associated with one or more areas of interest of the digitized image of the specimen are available for further operations. Additionally, displaying the snapshot icon includes displaying a snapshot tag, the snapshot tag indicating a number of the one or more snapshots. Additional details of the snapshot icon are described throughout the disclosure and are not repeated here.

At block 428, operations may include receiving an operation on the snapshot icon.

At block 430, operations may include displaying the one or more snapshots of the one or more areas of interest of the digitized image of the specimen upon receiving the operation on the snapshot icon.

Referring to FIG. 4F, block 408 may include the following.

At block 432, operations may include displaying an alert icon. The alert icon may indicate whether one or more alerts associated with one or more areas of interest of the digitized image of the specimen are available for further operations. Additionally, displaying the alert icon includes displaying an alert tag, the alert tag indicating a number of the one or more alerts. Additional details of the alert icon are described throughout the disclosure and are not repeated here.

At block 434, operations may include receiving an operation on the alert icon.

At block 436, operations may include displaying the one or more alerts upon receiving the operation on the alert icon.

Referring to FIG. 4G, block 408 may include the following.

At block 438, operations may include displaying a note icon. The note icon may indicate whether a note associated with the digitized image of the specimen is available for further operations. Additional details of the note icon are described throughout the disclosure and are not repeated here.

At block 440, operations may include receiving an operation on the note icon.

At block 442, operations may include displaying the note upon receiving the operation on the note icon.

Referring to FIG. 4H, block 408 may include the following.

At block 444, operations may include displaying an additional information icon. The additional information icon may indicate whether additional information associated with the digitized image of the specimen is available for further operations. Additional details of the additional information icon are described throughout the disclosure and are not repeated here.

At block 446, operations may include receiving an operation on the additional information icon.

At block 448, operations may include displaying the additional information upon receiving the operation on the additional information icon.

Referring to FIG. 4I, block 408 may include the following.

At block 450, operations may include displaying an action icon. The action icon may indicate whether a follow-up action associated with the digitized image of the specimen is required Additional details of the action icon are described throughout the disclosure and are not repeated here.

At block 452, operations may include receiving an operation on the action icon.

At block 454, operations may include, in response to receiving the operation on the action icon, switching the slide panel from a normal state to a hidden state when the slide panel is in the normal state; or switching the slide panel from the hidden state to the normal state when the slide panel is in the hidden state.

Referring to FIG. 4J, process 400 may include the following.

At block 456, operations may include determining whether the digitized image of the specimen has been opened.

At block 458, operations may include changing a background of the slide panel from a first background color to a second background color when determining that the digitized image of the specimen has been opened.

Referring to FIG. 4K, block 408 may include the following.

At block 460, operations may include displaying a collapse-expand icon. The collapse-expand icon may indicate whether the slide panel is in a collapsed view mode or an expanded view mode. Additional details of the collapse-expand icon are described throughout the disclosure and are not repeated here.

At block 462, operations may include receiving an operation on the collapse-expand icon.

At block 464, operations may include, in response to receiving the operation on the collapse-expand icon, switching the slide panel from the collapsed view mode to the expanded view mode when the slide panel is in the collapsed view mode; or switching the slide panel from the expanded view mode to the collapsed view mode when the slide panel is in the expanded view mode.

With the process 400, the one or more interactive icons enable the user to know the status associated with the slide panel without opening the digitized image of the specimen. Additionally, the one or more interactive icons are operable such that the user may view the data associated with the slide panel such as annotations, snapshots, alerts, notes, additional information, follow-up action requirements, and so on by operating the one or more interactive icons without opening the digitized image of the specimen. Additionally, various versions of the same application may be accessible to the user at the same time. Thus, the usability of the digital pathology platform is improved. The workflow of the user may be facilitated.

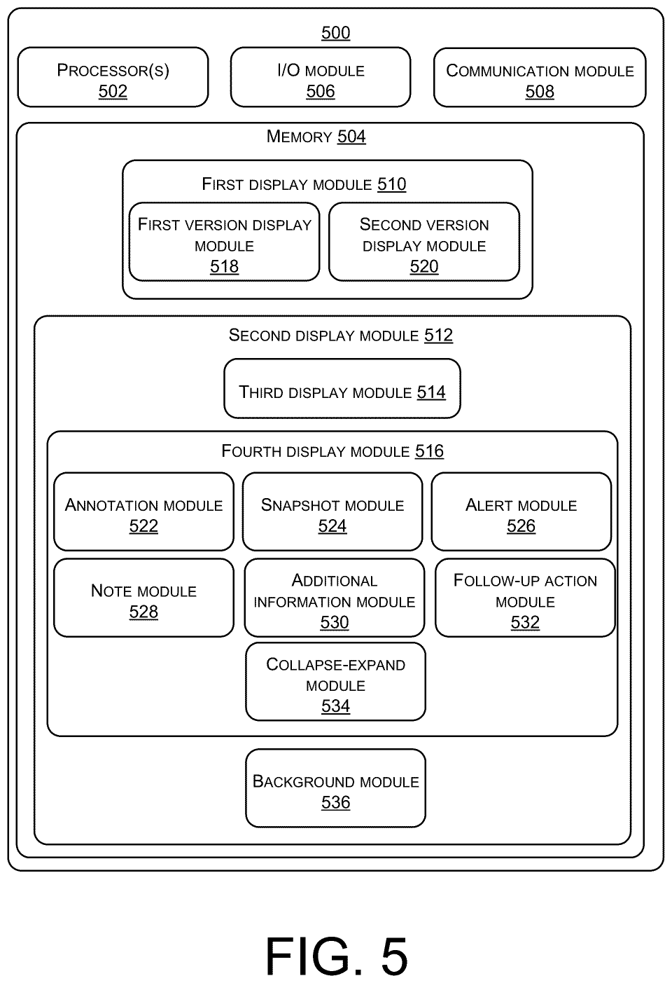

FIG. 5 illustrates an example apparatus 500 for implementing the processes and methods described above.

The techniques and mechanisms described herein may be implemented by multiple instances of the apparatus 500 as well as by any other computing device, system, and/or environment. The apparatus 500 shown in FIG. 5 is only one example of a system and is not intended to suggest any limitation as to the scope of use or functionality of any computing device utilized to perform the processes and/or procedures described above. Other well-known computing devices, systems, environments and/or configurations that may be suitable for use with the embodiments include, but are not limited to, personal computers, server computers, hand-held or laptop devices, multiprocessor systems, microprocessor-based systems, set top boxes, game consoles, programmable consumer electronics, network PCs, minicomputers, mainframe computers, distributed computing environments that include any of the above systems or devices, implementations using field programmable gate arrays (“FPGAs”) and application specific integrated circuits (“ASICs”), and/or the like.

The apparatus 500 may include one or more processors 502 and system memory 504 communicatively coupled to the processor(s) 502. The processor(s) 502 may execute one or more modules and/or processes to cause the processor(s) 502 to perform a variety of functions. In some embodiments, the processor(s) 502 may include a central processing unit (CPU), a graphics processing unit (GPU), both CPU and GPU, or other processing units or components known in the art. Additionally, each of the processor(s) 502 may possess its own local memory, which also may store program modules, program data, and/or one or more operating systems.

Depending on the exact configuration and type of the apparatus 500, the system memory 504 may be volatile, such as RAM, non-volatile, such as ROM, flash memory, miniature hard drive, memory card, and the like, or some combination thereof. The system memory 504 may include one or more computer-executable modules (modules) that are executable by the processor(s) 502.

The apparatus 500 may additionally include an input/output (I/O) interface 506 for receiving data to be processed, and for outputting the processed data. The apparatus 500 may also include a communication module 508 allowing the apparatus 500 to communicate with other devices (not shown) over a network (not shown). The network may include the Internet, wired media such as a wired network or direct-wired connections, and wireless media such as acoustic, radio frequency (RF), infrared, and other wireless media.

The one or more computer-executable modules may include the following.

A first display module 510 may be configured to display a user interface for a digital pathology platform.

A second display module 512 may be configured to display a slide panel on the user interface. Additional details of the slide panel are described throughout the disclosure and are not repeated here.

The second display module 512 may include a third display module 514 and a fourth display module 516.

The third display module 514 may be configured to display a thumbnail of a digitized image of a specimen. Additional details of the thumbnail are described throughout the disclosure and are not repeated here.

The fourth display module 516 may be configured to display one or more interactive icons associated with the slide panel. The one or more interactive icons indicating status associated with the slide panel.

The first display module 510 may include a first version display module 518 and a second version display module 520.

The first version display module 518 may be configured to display a first button of a first version of an application; receive an operation on the first button of the first version of the application; and display the first version of the application upon receiving the operation on the first button.

The second version display module 520 may be configured to display a second button of a second version of the application; receive an operation on the second button of the second version of the application; and display the second version of the application upon receiving the operation on the second button.

Additional details of the version switching of the application are described throughout the disclosure and are not repeated here.

The fourth display module 514 may include an annotation module 522, a snapshot module 524, an alert module 526, a note module 528, an additional information module 530, a follow-up action module 532, and a collapse-expand module 534.

The annotation module 522 may be configured to display an annotation icon. The annotation icon may indicate whether one or more annotations associated with one or more areas of interest of the digitized image of the specimen are available for further operations. Additionally, the annotation module 522 may be further configured to display an annotation tag, the annotation tag indicating a number of the one or more annotations. Additional details of the annotation icon are described throughout the disclosure and are not repeated here.

Additionally, the annotation module 522 may be further configured to receive an operation on the annotation icon; and display the one or more annotations associated with the one or more areas of interest of the digitized image of the specimen upon receiving the operation on the annotation icon.

The snapshot module 524 may be configured to display a snapshot icon. The snapshot icon may indicate whether one or more snapshots associated with one or more areas of interest of the digitized image of the specimen are available for further operations. Additionally, the snapshot module 524 is further configured to display a snapshot tag, the snapshot tag indicating a number of the one or more snapshots. Additional details of the snapshot icon are described throughout the disclosure and are not repeated here.

Additionally, the snapshot module 524 may be further configured to receive an operation on the snapshot icon; and display the one or more snapshots of the one or more areas of interest of the digitized image of the specimen upon receiving the operation on the snapshot icon.

The alert module 526 may be configured to display an alert icon. The alert icon may indicate whether one or more alerts associated with one or more areas of interest of the digitized image of the specimen are available for further operations. Additionally, the alert module 526 is further configured to display an alert tag, the alert tag indicating a number of the one or more alerts. Additional details of the alert icon are described throughout the disclosure and are not repeated here.

Additionally, the alert module 526 is further configured to receive an operation on the alert icon; and display the one or more alerts upon receiving the operation on the alert icon.

The note module 528 may be configured to a note icon. The note icon may indicate whether a note associated with the digitized image of the specimen is available for further operations. Additional details of the note icon are described throughout the disclosure and are not repeated here.

Additionally, the note module 528 may be further configured to receive an operation on the note icon; and display the note upon receiving the operation on the note icon.

The additional information module 530 may be configured to display an additional information icon. The additional information icon may indicate whether additional information associated with the digitized image of the specimen is available for further operations. Additional details of the additional information icon are described throughout the disclosure and are not repeated here.

Additionally, the additional information module 530 may be further configured to receive an operation on the additional information icon; and display the additional information upon receiving the operation on the additional information icon.

The follow-up action module 532 may be configured to display an action icon. The action icon may indicate whether a follow-up action associated with the digitized image of the specimen is required. Additional details of the action icon are described throughout the disclosure and are not repeated here.

Additionally, the follow-up action module 532 may be further configured to receive an operation on the action icon; and in response to receiving the operation on the action icon, switch the slide panel from a normal state to a hidden state when the slide panel is in the normal state, or switch the slide panel from the hidden state to the normal state when the slide panel is in the hidden state.

The collapse-expand module 534 may be configured to display a collapse-expand icon. The collapse-expand icon may indicate whether the slide panel is in a collapsed view mode or an expanded view mode. Additional details of the collapse-expand icon are described throughout the disclosure and are not repeated here.

Additionally, the collapse-expand module 534 may be further configured to receive an operation on the collapse-expand icon; and in response to receiving the operation on the collapse-expand icon, switch the slide panel from the collapsed view mode to the expanded view mode when the slide panel is in the collapsed view mode, or switch the slide panel from the expanded view mode to the collapsed view mode when the slide panel is in the expanded view mode.

The memory 504 may further include a background module 536, which may be configured to determine whether the digitized image of the specimen has been opened; and change a background of the slide panel from a first background color to a second background color when determining that the digitized image of the specimen has been opened.

With the apparatus 500, the one or more interactive icons enable the user to know the status associated with the slide panel without opening the digitized image of the specimen. Additionally, the one or more interactive icons are operable such that the user may view the data associated with the slide panel such as annotations, snapshots, alerts, notes, additional information, follow-up action requirements, and so on by operating the one or more interactive icons without opening the digitized image of the specimen. Additionally, various versions of the same application may be accessible to the user at the same time. Thus, the usability of the digital pathology platform may be improved. The workflow of the user may be facilitated.

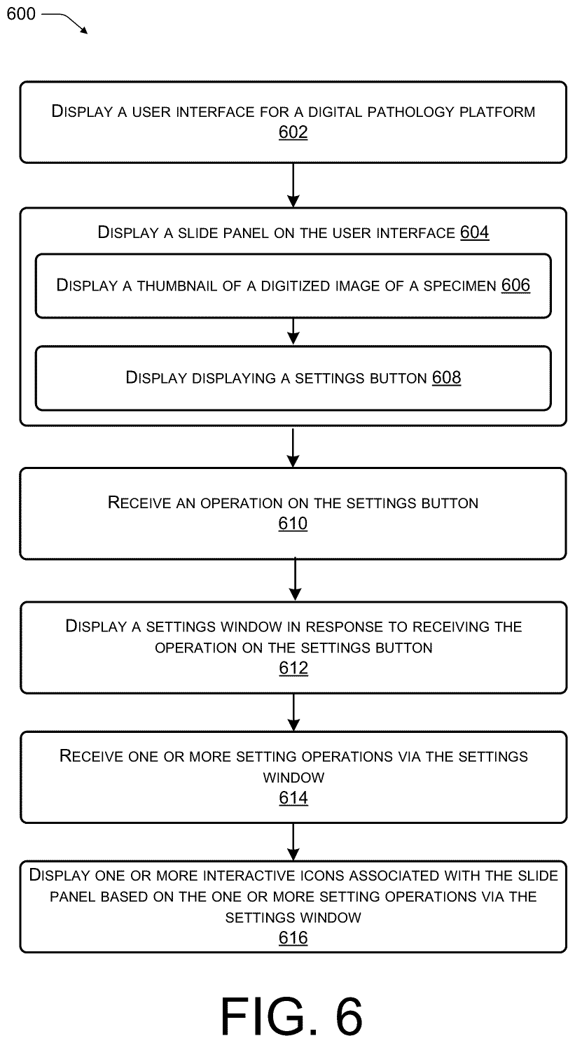

FIG. 6 illustrates an example process 600 for enhancing digital pathology platform according to implementations of this disclosure.

Referring to FIG. 6, process 600 may include the following.

At block 602, operations may include displaying a user interface for a digital pathology platform.

At block 604, operations may include displaying a slide panel on the user interface. Displaying the slide panel includes displaying a thumbnail of a digitized image of a specimen (block 606) and displaying a settings button (block 608).

At block 610, operations may include receiving an operation on the settings button.

At block 612, operations may include displaying a settings window in response to receiving the operation on the settings button.

In implementations, displaying the settings window in response to receiving the operation on the settings button can include displaying one or more operable fields configured to set the one or more interactive icons. The one or more operable fields can include, but are not limited to, an annotation field, a snapshot field, an alert field, a note field, an additional information field, or the like. More details regarding the operable fields are given throughout this disclosure.

At block 614, operations may include receiving one or more setting operations via the settings window.

In implementations, receiving the one or more setting operations via the settings window can include receiving a first setting operation in the annotation field, receiving a second setting operation in the snapshot field, receiving a third setting operation in the alert field, receiving a fourth setting operation in the note field, receiving a fifth setting operation in the additional information field, or the like. More details regarding the operable fields are given throughout this disclosure.

At block 616, operations may include displaying one or more interactive icons associated with the slide panel based on the one or more setting operations via the settings window, the one or more interactive icons indicating status associated with the slide panel.

In implementations, displaying the one or more interactive icons associated with the slide panel can include displaying/hiding the annotation icon based on the first setting operation in the annotation field, displaying/hiding the snapshot icon based on the second setting operation in the snapshot field, displaying/hiding the alert icon based on the third setting operation in the alert field, displaying/hiding the note icon based on the fourth setting operation in the note field, displaying/hiding the additional information icon based on the fifth setting operation in the additional information field, or the like. More details regarding the operable fields and the interactive icons are given throughout this disclosure.

Some or all operations of the methods described above may be performed by execution of computer-readable instructions stored on a computer-readable storage medium, as defined below. The term “computer-readable instructions” as used in the description and claims, includes routines, applications, application modules, program modules, programs, components, data structures, algorithms, and the like. Computer-readable instructions may be implemented on various system configurations, including single-processor or multiprocessor systems, minicomputers, mainframe computers, personal computers, hand-held computing devices, microprocessor-based, programmable consumer electronics, combinations thereof, and the like.

The computer-readable storage media may include volatile memory (such as random access memory (RAM)) and/or non-volatile memory (such as read-only memory (ROM), flash memory, etc.). The computer-readable storage media may also include additional removable storage and/or non-removable storage including, but is not limited to, flash memory, magnetic storage, optical storage, and/or tape storage that may provide non-volatile storage of computer-readable instructions, data structures, program modules, and the like.

A non-transient computer-readable storage medium is an example of computer-readable media. Computer-readable media includes at least two types of computer-readable media, namely computer-readable storage media and communications media. Computer-readable storage media includes volatile and non-volatile, removable and non-removable media implemented in any process or technology for storage of information such as computer-readable instructions, data structures, program modules, or other data. Computer-readable storage media includes, but is not limited to, phase-change memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disc read-only memory (CD-ROM), digital versatile discs (DVD) or other optical storage, magnetic cassettes, magnetic tape, magnetic disk storage or other magnetic storage devices, or any other non-transmission medium that may be used to store information for access by a computing device. In contrast, communication media may embody computer-readable instructions, data structures, program modules, or other data in a modulated data signal, such as a carrier wave, or other transmission mechanisms. As defined herein, computer-readable storage media do not include communication media.

The computer-readable instructions stored on one or more non-transitory computer-readable storage media that, when executed by one or more processors, may perform operations described above with reference to the drawings. Generally, computer-readable instructions include routines, programs, objects, components, data structures, and the like that perform particular functions or implement particular abstract data types. The order in which the operations are described is not intended to be construed as a limitation, and any number of the described operations may be omitted or combined in any order and/or in parallel to implement the processes.

### CONCLUSION

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

### Example Clauses

Clause 1. A method, comprising: displaying a user interface for a digital pathology platform; and displaying a slide panel on the user interface, wherein displaying the slide panel includes displaying a thumbnail of a digitized image of a specimen; and displaying one or more interactive icons associated with the slide panel, the one or more interactive icons indicating status associated with the slide panel.

Clause 2. The method of clause 1, wherein displaying the user interface for the digital pathology platform includes displaying a first button of a first version of an application and a second button of a second version of the application.

Clause 3. The method of clause 2, further comprising: receiving an operation on the first button of the first version of the application; and displaying the first version of the application upon receiving the operation on the first button.

Clause 4. The method of clause 2, further comprising: receiving an operation on the second button of the second version of the application; and displaying the second version of the application upon receiving the operation on the second button.

Clause 5. The method of clause 1, wherein displaying the one or more interactive icons associated with the slide panel includes displaying an annotation icon, the annotation icon indicating whether one or more annotations associated with one or more areas of interest of the digitized image of the specimen are available for further operations.

Clause 6. The method of clause 5, wherein displaying the annotation icon includes displaying an annotation tag, the annotation tag indicating a number of the one or more annotations.

Clause 7. The method of clause 5, further comprising: receiving an operation on the annotation icon; and displaying the one or more annotations associated with the one or more areas of interest of the digitized image of the specimen upon receiving the operation on the annotation icon.

Clause 8. The method of clause 1, wherein displaying the one or more interactive icons associated with slide panel includes displaying a snapshot icon, the snapshot icon indicating whether one or more snapshots of one or more areas of interest of the digitized image of the specimen are available for further operations.

Clause 9. The method of clause 8, wherein displaying the snapshot icon includes displaying a snapshot tag, the snapshot tag indicating a number of the one or more snapshots.

Clause 10. The method of clause 8, further comprising: receiving an operation on the snapshot icon; and displaying the one or more snapshots of the one or more areas of interest of the digitized image of the specimen upon receiving the operation on the snapshot icon.

Clause 11. The method of clause 1, wherein displaying the one or more interactive icons associated with slide panel includes displaying an alert icon, the alert icon indicating whether one or more alerts associated with the digitized image of the specimen are available for further operations.

Clause 12. The method of clause 11, wherein displaying the alert icon includes displaying an alert tag, the alert tag indicating a number of the one or more alerts.

Clause 13. The method of clause 11, further comprising: receiving an operation on the alert icon; and displaying the one or more alerts upon receiving the operation on the alert icon.

Clause 14. The method of clause 1, wherein displaying the one or more interactive icons associated with the slide panel includes displaying a note icon, the note icon indicating whether a note associated with the digitized image of the specimen is available for further operations.

Clause 15. The method of clause 14, further comprising: receiving an operation on the note icon; and displaying the note upon receiving the operation on the note icon.

Clause 16. The method of clause 1, wherein displaying the one or more interactive icons associated with the slide panel includes displaying an additional information icon, the additional information icon indicating whether additional information associated with the digitized image of the specimen is available for further operations.

Clause 17. The method of clause 16, further comprising: receiving an operation on the additional information icon; and displaying the additional information upon receiving the operation on the additional information icon.

Clause 18. The method of clause 1, wherein displaying the one or more interactive icons associated with slide panel includes displaying an action icon, the action icon indicating whether a follow-up action associated with the digitized image of the specimen is required.

Clause 19. The method of clause 18, further comprising: receiving an operation on the action icon; and in response to receiving the operation on the action icon, switching the slide panel from a normal state to a hidden state when the slide panel is in the normal state; or switching the slide panel from the hidden state to the normal state when the slide panel is in the hidden state.

Clause 20. The method of clause 1, further comprising: determining whether the digitized image of the specimen has been opened; and changing a background of the slide panel from a first background color to a second background color when determining that the digitized image of the specimen has been opened.

Clause 21. The method of clause 1, wherein displaying the one or more interactive icons associated with the slide panel includes displaying a collapse-expand icon, the collapse-expand icon indicating whether the slide panel is in a collapsed view mode or an expanded view mode.

Clause 22. The method of clause 21, further comprising: receiving an operation on the collapse-expand icon; and in response to receiving the operation on the collapse-expand icon, switching the slide panel from the collapsed view mode to the expanded view mode when the slide panel is in the collapsed view mode; or switching the slide panel from the expanded view mode to the collapsed view mode when the slide panel is in the expanded view mode.

Clause 23. An apparatus, comprising: one or more processors; memory, coupled to the one or more processors, the memory storing thereon computer-executable modules, executable by the one or more processors, the computer-executable modules including a first display module configured to display a user interface for a digital pathology platform; a second display module configured to display a slide panel on the user interface, wherein the second display module includes a third display module configured to display a thumbnail of a digitized image of a specimen on the user interface; and a fourth display module configured to display one or more interactive icons associated with the slide panel, the one or more interactive icons indicating status associated with the slide panel.

Clause 24. One or more computer-readable media, stored thereon computer-readable instructions that, when executed by one or more processors, cause the one or more processors to perform acts comprising: displaying a user interface for a digital pathology platform; and displaying a slide panel on the user interface, wherein displaying the slide panel includes displaying a thumbnail of a digitized image of a specimen; and displaying one or more interactive icons associated with the slide panel, the one or more interactive icons indicating status associated with the slide panel.

Clause 25. The one or more computer-readable media of clause 24, wherein displaying the user interface for the digital pathology platform includes displaying a first button of a first version of an application and a second button of a second version of the application.

What is claimed is:

1. A method, comprising:
displaying a user interface for a digital pathology platform; and
displaying a slide panel on the user interface, wherein displaying the slide panel includes:
displaying a thumbnail of a digitized image of a specimen; and
displaying a settings button;
receiving an operation on the settings button;
displaying a settings window in response to receiving the operation on the settings button;
receiving one or more setting operations via the settings window; and
displaying one or more interactive icons associated with the slide panel based on the one or more setting operations via the settings window, the one or more interactive icons indicating status associated with the slide panel.

2. The method of claim 1, wherein displaying the settings window in response to receiving the operation on the settings button comprises:
displaying one or more operable fields configured to set the one or more interactive icons.

3. The method of claim 2, wherein:
displaying the one or more operable fields comprises displaying an annotation field configured to set an annotation icon; and
receiving the one or more setting operations via the settings window comprises receiving a first setting operation in the annotation field.

4. The method of claim 3, wherein displaying the one or more interactive icons associated with the slide panel comprises at least one of:
displaying the annotation icon based on the first setting operation in the annotation field, the annotation icon indicating whether one or more annotations associated with one or more areas of interest of the digitized image of the specimen are available for further operations; or
hiding the annotation icon based on the first setting operation in the annotation field.

5. The method of claim 2, wherein:
displaying the one or more operable fields comprises displaying a snapshot field configured to set a snapshot icon; and
receiving the one or more setting operations via the settings window comprises receiving a second setting operation in the snapshot field.

6. The method of claim 5, wherein displaying the one or more interactive icons associated with the slide panel comprises at least one of:
displaying the snapshot icon based on the second setting operation in the snapshot field, the snapshot icon indicating whether one or more snapshots of one or more areas of interest of the digitized image of the specimen are available for further operations; or
hiding the snapshot icon based on the second setting operation in the snapshot field.

7. The method of claim 2, wherein:
displaying the one or more operable fields comprises displaying an alert field configured to set an alert icon; and
receiving the one or more setting operations via the settings window comprises receiving a third setting operation in the alert field.

8. The method of claim 7, wherein displaying the one or more interactive icons associated with the slide panel comprises at least one of:
displaying the alert icon based on the third setting operation in the alert field, the alert icon indicating whether one or more alerts associated with the digitized image of the specimen are available for further operations; or
hiding the alert icon based on the third setting operation in the alert field.

9. The method of claim 2, wherein:
displaying the one or more operable fields comprises displaying a note field configured to set a note icon; and
receiving the one or more setting operations via the settings window comprises receiving a fourth setting operation in the note field.

10. The method of claim 9, wherein displaying the one or more interactive icons associated with the slide panel comprises at least one of:
displaying the note icon based on the fourth setting operation in the note field, the note icon indicating whether a note associated with the digitized image of the specimen is available for further operations; or
hiding the note icon based on the fourth setting operation in the note field.

11. The method of claim 2, wherein:
displaying the one or more operable fields comprises displaying an additional information field configured to set an additional information icon; and
receiving the one or more setting operations via the settings window comprises receiving a fifth setting operation in the additional information field.

12. The method of claim 11, wherein displaying the one or more interactive icons associated with the slide panel comprises at least one of:
displaying the additional information icon based on the fifth setting operation in the additional information field, the additional information icon indicating whether additional information associated with the digitized image of the specimen is available for further operations; or
hiding the additional information icon based on the fifth setting operation in the additional information field.

13. One or more computer-readable media, stored thereon computer-readable instructions that, when executed by one or more processors, cause the one or more processors to perform acts comprising:
displaying a user interface for a digital pathology platform; and
displaying a slide panel on the user interface, wherein displaying the slide panel includes:
displaying a thumbnail of a digitized image of a specimen; and
displaying a settings button;
receiving an operation on the settings button;
displaying a settings window in response to receiving the operation on the settings button;
receiving one or more setting operations via the settings window; and
displaying one or more interactive icons associated with the slide panel based on the one or more setting operations via the settings window, the one or more interactive icons indicating status associated with the slide panel.

14. The one or more computer-readable media of claim 13, wherein displaying the settings window in response to receiving the operation on the settings button comprises:
displaying one or more operable fields configured to set the one or more interactive icons in the settings window.

15. The one or more computer-readable media of claim 14, wherein:
displaying the one or more operable fields comprises displaying an annotation field configured to set an annotation icon; and
receiving the one or more setting operations via the settings window comprises receiving a first setting operation in the annotation field.

16. The one or more computer-readable media of claim 15, wherein displaying the one or more interactive icons associated with the slide panel comprises at least one of:
displaying the annotation icon based on the first setting operation in the annotation field, the annotation icon indicating whether one or more annotations associated with one or more areas of interest of the digitized image of the specimen are available for further operations; or
hiding the annotation icon based on the first setting operation in the annotation field.

17. The one or more computer-readable media of claim 14, wherein:
displaying the one or more operable fields comprises displaying a snapshot field configured to set a snapshot icon; and
receiving the one or more setting operations via the settings window comprises receiving a second setting operation in the snapshot field.

18. The one or more computer-readable media of claim 17, wherein displaying the one or more interactive icons associated with the slide panel comprises at least one of:
displaying the snapshot icon based on the second setting operation in the snapshot field, the snapshot icon indicating whether one or more snapshots of one or more areas of interest of the digitized image of the specimen are available for further operations; or
hiding the snapshot icon based on the second setting operation in the snapshot field.

19. An apparatus, comprising:
one or more processors;
memory, coupled to the one or more processors, the memory storing thereon computer-readable instructions that, when executed by one or more processors, cause the one or more processors to perform acts comprising:
displaying a user interface for a digital pathology platform; and
displaying a slide panel on the user interface, wherein displaying the slide panel includes:
displaying a thumbnail of a digitized image of a specimen; and
displaying a settings button;
receiving an operation on the settings button;
displaying a settings window in response to receiving the operation on the settings button;
receiving one or more setting operations via the settings window; and
displaying one or more interactive icons associated with the slide panel based on the one or more setting operations via the settings window, the one or more interactive icons indicating status associated with the slide panel.

20. The apparatus of claim 19, wherein displaying the settings window in response to receiving the operation on the settings button comprises:
displaying one or more operable fields configured to set the one or more interactive icons in the settings window.
